(19) United States
(12) Patent Application Publication
(10) Pub. No.: US 2008/0262074 A1
(43) Pub. Date: Oct. 23, 2008

(54) Salt Forms of [R-(R*,R*)]-2-(4-Fluorophenyl)-Beta,Delta-Dihydroxy-5-(1-Methylethyl)-3-(Phenyl-4-[(Phenylamino)Carbonyl]-1H-Pyrrole-1-Heptanoic Acid

(75) Inventors: George Quallich, North Stonington, CT (US); Peter Pawloy, Perchtoldsdorf (AU)

Correspondence Address: PFIZER INC., PATENT DEPARTMENT, MS8260-1611, EASTERN POINT ROAD, GROTON, CT, 06340, US

(73) Assignee: PFIZER, INC., New York, NY (US)

(21) Appl. No.: 11/569,511

(22) PCT Filed: May 13, 2005
(86) PCT No.: PCT/IB05/01473

§ 371 Date: Sep. 4, 2007

Related U.S. Application Data
(60) Provisional application No. 60/573,812, filed on May 24, 2004.

(51) Int. Cl. A61K 31/40; C07D 207/337; C07D 207/34; A61P 25/28

(52) U.S. Cl. 514/427; 548/561; 548/537

(57) ABSTRACT

Novel salt forms of [R—(R*,R*)]-2-(4-fluorophenyl)-β,δ-dihydroxy-5-(1-methylethyl)-3-phenyl-4-[(phenylamino)carbonyl]-1H-pyrrole-1-heptanoic acid or solvates or hydrates thereof, as well as crystalline salts characterized by their X-ray powder diffraction pattern are described, as well as methods for the preparation and pharmaceutical composition of the same, which are useful as agents for treating hyperlipidemia, hypercholesterolemia, osteoporosis, benign prostatic hyperplasia, and Alzheimer's Disease.

### FIELD OF THE INVENTION

The present invention relates to novel salt forms of atorvastatin which is known by the chemical name [R—(R*,R*)]-2-(4-fluorophenyl)-β,δ-dihydroxy-5-(1-methylethyl)-3-phenyl-4-[(phenylamino)carbonyl]-1H-pyrrole-1-heptanoic acid, useful as pharmaceutical agents, to methods for their production and isolation to pharmaceutical compositions which include these compounds and a pharmaceutically acceptable carrier, as well as methods of using such compositions to treat subjects, including human subjects, suffering from hyperlipidemia, hypercholesterolemia, benign prostatic hyperplasia, osteoporosis, and Alzheimer's Disease.

### BACKGROUND OF THE INVENTION

The conversion of 3-hydroxy-3-methylglutaryl-coenzyme A (HMG-CoA) to mevalonate is an early and rate-limiting step in the cholesterol biosynthetic pathway. This step is catalyzed by the enzyme HMG-CoA reductase. Statins inhibit HMG-CoA reductase from catalyzing this conversion. As such, statins are collectively potent lipid lowering agents.

Atorvastatin calcium is currently sold as Lipitor® having the chemical name [R—(R*,R*)]-2-(4-fluorophenyl)-β,δ-dihydroxy-5-(1-methylethyl)-3-phenyl-4-[(phenylamino)carbonyl]-1H-pyrrole-1-heptanoic acid calcium salt (2:1) trihydrate and the formula:

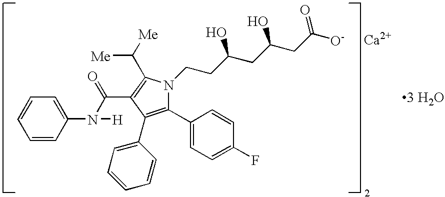

The nonproprietary name designated by USAN (United States Adopted Names) is atorvastatin calcium and by INN (International Nonproprietary Name) is atorvastatin. Under the established guiding principles of USAN, the salt is included in the name whereas under INN guidelines, a salt description is not included in the name.

Atorvastatin calcium is a selective, competitive inhibitor of HMG-CoA reductase. As such, atorvastatin calcium is a potent lipid lowering compound and is thus useful as a hypolipidemic and/or hypocholesterolemic agent, as well as in the treatment of osteoporosis, benign prostatic hyperplasia, and Alzheimer's disease.

A number of patents have issued disclosing atorvastatin calcium, formulations of atorvastatin calcium, as well as processes and key intermediates for preparing atorvastatin calcium. These include: U.S. Pat. Nos. 4,681,893; 5,273,995; 5,003,080; 5,097,045; 5,103,024; 5,124,482; 5,149,837; 5,155,251; 5,216,174; 5,245,047; 5,248,793; 5,280,126; 5,397,792; 5,342,952; 5,298,627; 5,446,054; 5,470,981; 5,489,690; 5,489,691; 5,510,488; 5,686,104; 5,998,633; 6,087,511; 6,126,971; 6,433,213; and 6,476,235, which are herein incorporated by reference.

Atorvastatin calcium can exist in crystalline, liquid-crystalline, non-crystalline and amorphous forms.

Crystalline forms of atorvastatin calcium are disclosed in U.S. Pat. Nos. 5,969,156, 6,121,461, and 6,605,729 which are herein incorporated by reference.

Additionally, a number of published International patent applications have disclosed crystalline forms of atorvastatin calcium, as well as processes for preparing amorphous atorvastatin calcium. These include: WO 00/71116; WO 01/28999; WO 01/36384; WO 01/42209; WO 02/41834; WO 02/43667; WO 02/43732; WO 02/051804; WO 02/057228; WO 02/057229; WO 02/057274; WO 02/059087; WO 02/072073; WO 02/083637; WO 02/083638; WO 02/089788; WO 03/050085; WO 03/070702; and WO 04/022053.

Atorvastatin is prepared as its hemi-calcium salt, i.e., [R—(R*,R*)]-2-(4-fluorophenyl)-β,δ-dihydroxy-5-(1-methylethyl)-3-phenyl-4-[(phenylamino)carbonyl]-1H-pyrrole-1-1-heptanoic acid calcium salt (2:1). The hemi-calcium salt is desirable since it enables atorvastatin to be conveniently formulated in, for example, tablets, capsules, lozenges, powders, and the like for oral administration.

U.S. Pat. No. 5,273,995 discloses the mono-sodium, mono-potassium, hemi-calcium, N-methylglucamine, hemi-magnesium, hemi-zinc, and the 1-deoxy-1-(methylamino)-D-glucitol (N-methylglucamine) salts of atorvastatin.

Also, atorvastatin free acid, disclosed in U.S. Pat. No. 5,273,995, can be used to prepare these salts of atorvastatin.

Additionally, U.S. Pat. No. 6,583,295 B1 discloses a series of amine salts of HMG-CoA reductase inhibitors which are used in a process for isolation and/or purification of these HMG-CoA reductase. The tertiary butylamine and dicyclohexylamine salts of atorvastatin are disclosed.

Co-pending United States patent application commonly owned, attorney case number PC25265, Ser. No. 60/568,379, discloses a series of novel salt forms of atorvastatin.

We have now surprisingly and unexpectedly found novel salt forms of atorvastatin including salts with alkaline earth metals or zinc. In particular, mono-alkaline earth metal salts of atorvastatin, including mono-calcium, have desirable properties. For example, the mono-alkaline earth metal salts of atorvastatin are anticipated to have increased aqueous solubility over the hemi-calcium salt of atorvastatin because of the decreased molecular weight of the former. This decrease in molecular weight facilitates an augmented intrinsic solubility. Increased aqueous solubility often translates to higher bioavailability in humans. As such, these salt forms are pharmaceutically acceptable and can be used to prepare pharmaceutical formulations. Thus, the present invention provides basic mono-alkaline earth metal or zinc salts of atorvastatin that are pure, have good stability, and have advantageous formulation properties compared to prior salt forms of atorvastatin.

### SUMMARY OF THE INVENTION

Accordingly, a first aspect of the invention is directed to a compound of Formula I or a solvate or hydrate thereof

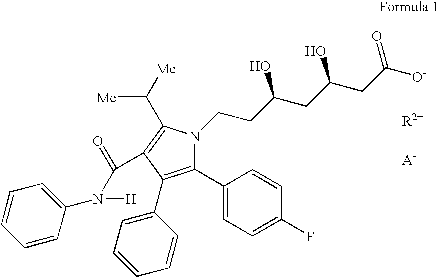

wherein R²⁺ is an alkaline earth metal or zinc and A⁻ is an anion.

In a second aspect, the invention is directed to a crystalline form of a compound of Formula I or a solvate or hydrate thereof.

In a third aspect, the invention is directed to a compound of Formula Ia or a solvate or hydrate thereof

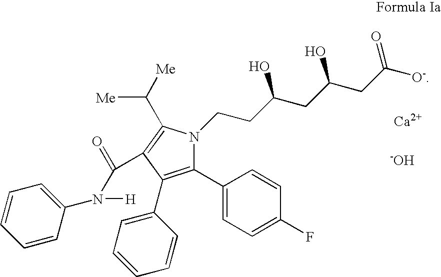

In a fourth aspect, the invention is directed to a crystalline form of a compound of Formula Ia or a solvate or hydrate thereof.

In a fifth aspect, the invention is directed to a compound of Formula Ib or a solvate or hydrate thereof

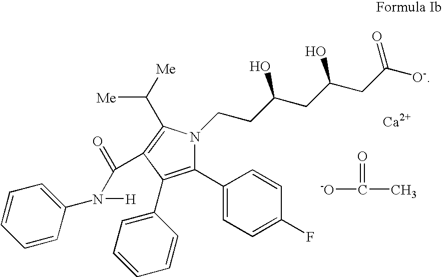

In a sixth aspect, the invention is described to a compound of Formula Ic or a solvate or hydrate thereof

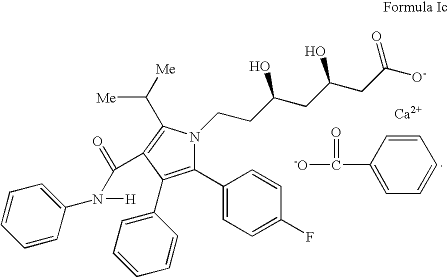

As inhibitors of HMG-CoA reductase, the novel salt forms of atorvastatin are useful as hypolipidemic and hypocholesterolemic agents, as well as agents in the treatment of osteoporosis, benign prostatic hyperplasia, and Alzheimer's Disease.

A still further embodiment of the present invention is a pharmaceutical composition for administering an effective amount of an atorvastatin salt in unit dosage form in the treatment methods mentioned above. Finally, the present invention is directed to methods for production of salt forms of atorvastatin.

### BRIEF DESCRIPTION OF THE DRAWING

The invention is further described by the following non-limiting examples which refer to the accompanying FIG. 1, short particulars of which are given below.

FIG. 1. Diffractogram of atorvastatin mono-calcium hydroxide carried out on a Bruker D5000 diffractometer.

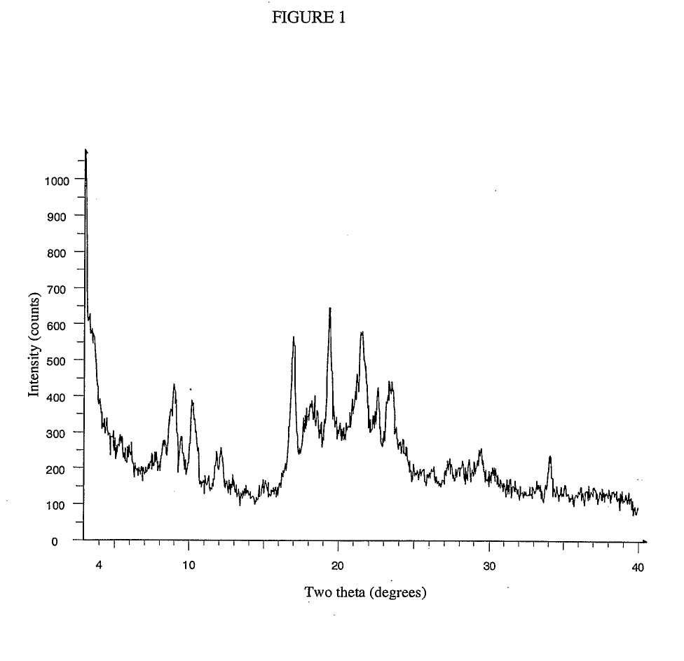

### DETAILED DESCRIPTION OF THE INVENTION

The novel salt forms of atorvastatin may be characterized by their x-ray powder diffraction patterns and/or by their solid-state nuclear magnetic resonance spectra.

### Powder X-ray Diffraction

Atorvastatin salts are characterized by their powder x-ray diffraction patterns. Thus, the x-ray diffraction pattern was carried out on a Bruker D5000 diffractometer using copper radiation (CuKₐ). The tube voltage and amperage are set to 40 kV and 50 mA, respectively. The divergence and scattering slits were set at 1 mm, and the receiving slit is set at 0.6 mm. Diffracted radiation was detected by a Kevex PSI detector. A theta-two theta continuous scan at 2.4°/min (1 sec/0.04° step) from 3.0 to 40°2θ was used. An alumina standard was analyzed to check the instrument alignment. Data were collected and analyzed using Bruker axis software Version 7.0. Samples were prepared for analysis by placing them in a quartz holder. It should be noted that Bruker Instruments purchased Siemans; thus, a Bruker D5000 instrument is essentially the same as a Siemans D5000.

### Solid State Nuclear Magnetic Resonance

The novel salt forms of atorvastatin may also be characterized by their solid-state nuclear magnetic resonance spectra (SSNMR), such as, for example, ¹⁹F SSNMR and ¹³C SSNMR.

In this invention, the term “alkaline earth metal” is a metal in Group IIA of the periodic table and includes, for example, calcium, magnesium, barium, strontium, and the like.

The term “anion” refers to an ion having a negative charge and includes, for example, OH⁻, R¹CO₂⁻ wherein R¹ is alkyl having from one to twelve carbon atoms or aryl and the like.

In this invention the term “alkyl” means a straight or branched hydrocarbon radical having from one to twelve carbon atoms and includes, for example, methyl, ethyl, n-propyl, isopropyl, n-butyl, secondary-butyl, isobutyl, tertiary butyl, n-pentyl, n-hexyl, n-heptyl, n-octyl, n-nonyl, n-decyl, undecyl, and dodecyl.

“Aryl” means a aromatic radical which is a phenyl group or a phenyl group substituted by one to three substituents selected from the group consisting of alkyl of from one to three carbon atoms, halogen, and nitro.

“Halogen” is iodine, bromine, chlorine, and fluorine.

“Solvate” refers to any pharmaceutical acceptable solvent, such as, for example, ethanol, acetic acid, and the like.

The crystalline salt forms of atorvastatin of the present invention, regardless of the extent of hydration and/or solvation having equivalent x-ray powder diffractograms, or SSNMR, are within the scope of the present invention.

The new salt forms of atorvastatin described herein have advantageous properties. For example, the mono salt of an alkaline earth metal or zinc salt of atorvastatin compared to a hemi salt of an alkaline earth metal or zinc salt of atorvastatin provides less forms of atorvastatin which reduces the complexity and increases the robustness of crystallization of the desired form. Thus, simplifying manufacturing and quality control. Additionally, the mono-alkaline earth metal salts of atorvastatin are anticipated to have increased water solubility over the hemi-calcium salt of atorvastatin because of the decreased molecular weight of the former. This decrease in molecular weight facilitates an augmented intrinsic solubility. Increased aqueous solubility often translates to higher bioavailability in humans.

The present invention provides a process for the preparation of the salt forms of atorvastatin which comprises preparing a solution of atorvastatin free acid (U.S. Pat. No. 5,213,995) in a solvent, such as, for example: methyl tertiary butyl ether, methanol, ethanol, isopropanol, acetone, water, and the like. The cationic counterion solutions or suspensions are prepared using 1.0 to about 1.2 equivalent in the same solvent. Water is added to some counterions to increase their solubility. The atorvastatin free acid solution is added to the counterion solution or suspension while stirring. The reaction is stirred for about 10 to about 72 hours at about ambient temperature to about 80° C. The reactions containing solids are vacuum filtered, washed with the reaction solvent, and air-dried overnight at ambient conditions. If precipitation is not present after ˜2 weeks, the solution is slowly evaporated. All salts are stored at ambient temperature and characterized as described above.

The compounds of the present invention can be prepared and administered in a wide variety of oral and parenteral dosage forms. Thus, the compounds of the present invention can be administered by injection, that is, intravenously, intramuscularly, intracutaneously, subcutaneously, intraduodenally, or intraperitoneally. Also, the compounds of the present invention can be administered by inhalation, for example, intranasally. Additionally, the compounds of the present invention can be administered transdermally.

For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substances which may also act as diluents, flavoring agents, solubilizers, lubricants, suspending agents, binders, preservatives, tablet disintegrating agents, or an encapsulating material.

In powders, the carrier is a finely divided solid which is in a mixture with the finely divided active component.

In tablets, the active component is mixed with the carrier having the necessary binding properties in suitable proportions and compacted in the shape and size desired.

The powders and tablets preferably contain from two or ten to about seventy percent of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa buffer, and the like. The term “preparation” is intended to include the formulation of the active compound with encapsulating material as a carrier providing a capsule in which the active component, with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogenous mixture is then poured into convenient sized molds, allowed to cool, and thereby to solidify.

Liquid form preparations include solutions, suspensions, retention enemas, and emulsions, for example water or water propylene glycol solutions. For parenteral injection, liquid preparations can be formulated in solution in aqueous polyethylene glycol solution.

Aqueous solutions suitable for oral use can be prepared by dissolving the active component in water and adding suitable colorants, flavors, stabilizing, and thickening agents as desired.

Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 0.5 mg to 100 mg, preferably 2.5 mg to 80 mg according to the particular application and the potency of the active component. The composition can, if desired, also contain other compatible therapeutic agents.

In therapeutic use as hypolipidemic and/or hypocholesterolemic agents and agents to treat osteoporosis, benign prostatic hyperplasia, and Alzheimer's disease, the salt forms of atorvastatin utilized in the pharmaceutical method of this invention are administered at the initial dosage of about 2.5 mg to about 80 mg daily. A daily dose range of about 2.5 mg to about 20 mg is preferred. The dosages, however, may be varied depending upon the requirements of the patient, the severity of the condition being treated, and the compound being employed. Determination of the proper dosage for a particular situation is within the skill of the art. Generally, treatment is initiated with smaller dosages which are less than the optimum dose of the compound. Thereafter, the dosage is increased by small increments until the optimum effect under the circumstance is reached. For convenience, the total daily dosage may be divided and administered in portions during the day if desired.

The following nonlimiting examples illustrate the inventors' preferred methods for preparing the compounds of the invention.

### EXAMPLE 1

### [R—(R*,R*)]-2-(4-Fluorophenyl)-β,δ-dihydroxy-5-(1-methylethyl)-3-phenyl-4-[(phenylamino)carbonyl]-1H-pyrrole-1-heptanoic acid, calcium salt (1:1) (atorvastatin mono-calcium hydroxide)

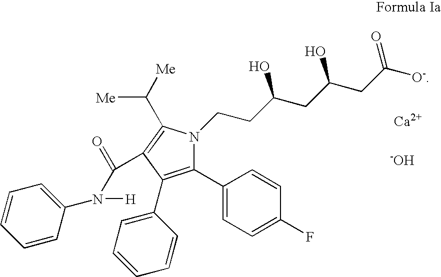

### Method A

A 25 mL reaction tube was charged with water (0.5 mL) and calcium hydroxide (13.2 mg, 0.000178 moles, 1.0 equiv). The mixture was stirred at 20 to 25° C. A solution of atorvastatin free acid (U.S. Pat. No. 5,273,995) (100 mg, 0.000178 moles, 1.0 equiv) dissolved in acetone (0.5 mL) was added dropwise by pipette over about 2 minutes into the aqueous suspension. The resulting slurry was stirred at 20 to 25° C. for about 48 hours under a nitrogen blanket. Water (2.0 mL) was added to the slurry and the mixture was allowed to stir for an additional 2 hours. The product solids were isolated by filtration on a paper filter covered Buchner funnel. The solids were washed twice with room temperature water (2×2 mL). The solids were dried under vacuum at 50 to 55° C. in an oven to yield atorvastatin mono-calcium hydroxide as a white solid (75 mg, 68% yield).

### Method B

A 25 mL reaction tube was charged with water (1.3 mL) and calcium hydroxide (13.3 mg, 0.00018 moles, 1.0 equiv). Atorvastatin free acid (U.S. Pat. No. 5,273,995) (100 mg, 0.00018 moles, 1.0 equiv.) and methanol (3 mL) were added to give a thick suspension. The resulting slurry was heated to reflux. The suspension was evaporated to dryness under vacuum on a rotary evaporator to afford a white solid. The solid was dried in a vacuum oven at 50 to 55° C. to yield atorvastatin mono-calcium hydroxide as a white solid (84.9 mg, 88% yield).

### Method C

The free acid of atorvastatin (U.S. Pat. No. 5,273,995) (1.0 g) is dissolved in methyl t-butyl ether (25 ml) and added dropwise to a solution of calcium acetate (311 mg) in water (15 mL) and isopropanol (10 mL). Sodium hydroxide (158 mg) in water (1.0 mL) is added and the contents are stirred for 24 hours. The methyl t-butyl ether is removed under vacuum and the resulting solid is filtered and in water (10 mL) for 24 hours. The solid is dried under vacuum to yield atorvastatin mono-calcium hydroxide.

### Method D

The free acid of atorvastatin (U.S. Pat. No. 5,273,995) (1.0 g) is added to a solution of water (10 mL) and sodium hydroxide (144 mg) at ambient temperature. After stirring for 30 minutes a solution of calcium chloride (200 mg) in water (20 mL) is added dropwise over three hours. The temperature is then raised to 50° to 80° and the contents vigorously stirred for 18 hours. Upon cooling the solid is filtered and dried to provide atorvastatin mono-calcium hydroxide.

### EXAMPLE 2

### [R—(R*,R*)]-2-(4-Fluorophenyl)-β,δ-dihydroxy-5-(1-methylethyl)-3-phenyl-4-[(phenylamino)carbonyl]-1H-pyrrole-1-heptanoic acid, calcium salt (1:1) (atorvastatin mono-calcium acetate)

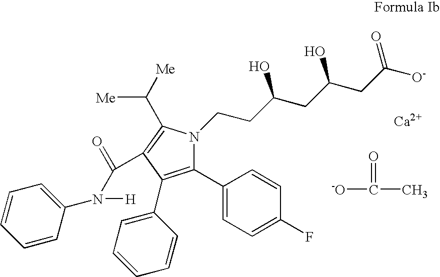

### Method A

Calcium hydroxide (133 mg) is suspended in ethanol (15 mL) and water (10 mL) and stirred for 15 minutes. The free acid of atorvastatin (U.S. Pat. No. 5,273,995) (1.0 g) is added to this suspension followed by 107 mg of acetic acid. The reaction is stirred for 5 hours at 25° C. and evaporated to dryness under vacuum to afford atorvastatin mono-calcium acetate.

### Method B

Calcium hydroxide (133 mg) is suspended in acetic acid (1 mL) and water (20 mL) and stirred for 15 minutes. The free acid of atorvastatin (U.S. Pat. No. 5,273,995) (1.0 g) is added to this suspension. The reaction is stirred for 5 hours at 25° C. and evaporated to dryness under vacuum to afford atorvastatin mono-calcium acetate.

### EXAMPLE 3

### [R—(R*,R*)]-2-(4-Fluorophenyl)-β,δ-dihydroxy-5-(1-methylethyl)-3-phenyl-4-[(phenylamino)carbonyl]-1H-pyrrole-1-heptanoic acid, calcium salt (1:1) (atorvastatin mono-calcium benzoate)

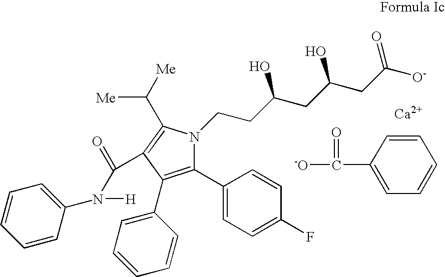

Calcium hydroxide (133 mg) is suspended in acetone (15 mL) and water (10 mL) and stirred for 15 minutes. The free acid of atorvastatin (U.S. Pat. No. 5,273,995) (1.0 g) is added to this suspension followed by 218 mg of benzoic acid. The reaction is stirred for 5 hours at 25° C. and evaporated to dryness under vacuum to afford atorvastatin mono-calcium benzoate.

What is claimed is:

1. A compound of Formula I or a solvate or hydrate thereof. 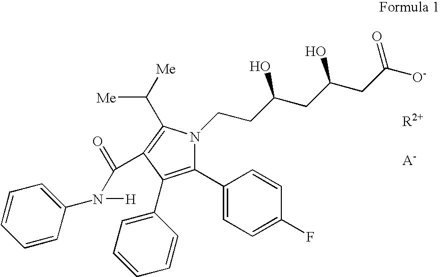
wherein R²⁺ is an alkaline earth metal or zinc and A⁻ is an anion.

2. The compound of claim 1 or a solvate or hydrate thereof wherein R²⁺ is selected from the group consisting of: calcium, magnesium, strontium, barium, and zinc.

3. The compound of claim 1 or a solvate or hydrate thereof wherein A⁻ is hydroxide or R¹CO₂⁻ wherein R¹ is alkyl having from one to twelve carbon atoms or aryl.

4. The compound of claim 2 or a solvate hydrate thereof wherein R²⁺ is calcium.

5. A crystalline form of a compound of Formula I or a solvate hydrate thereof 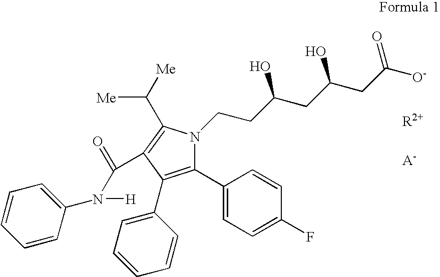
wherein R²⁺ is an alkaline earth metal or zinc and A⁻ is an anion.

6. A compound of Formula Ia or a solvate or hydrate thereof

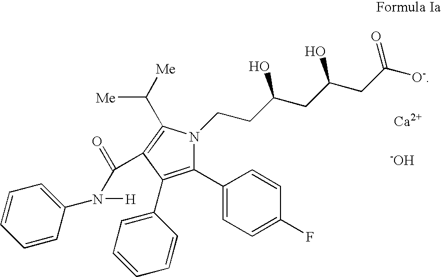

7. A compound selected from the group consisting of: a compound of Formula Ib or a solvate or hydrate thereof: 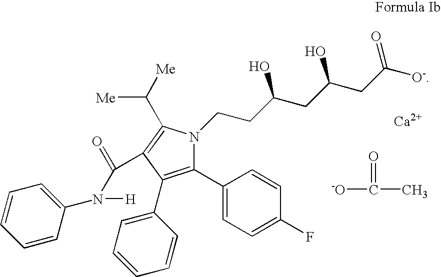
and a compound of Formula Ic or a solvate or hydrate thereof

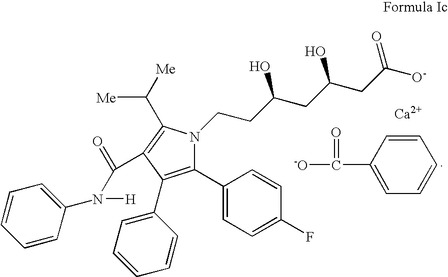

8. A crystalline form of a compound of Formula Ia or a solvate or hydrate thereof

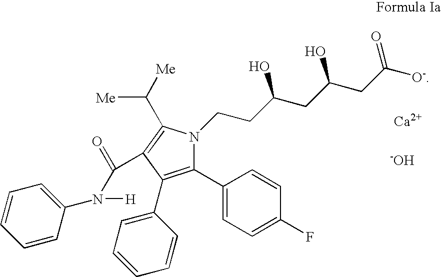

9. A pharmaceutical composition comprising a compound of Formula 1 or a solvate or hydrate thereof 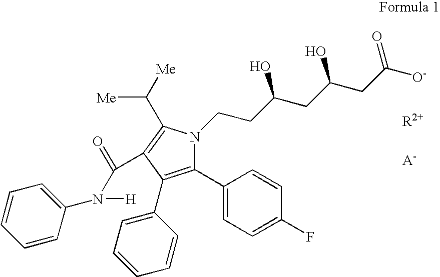
wherein R²⁺ is an alkaline earth metal or zinc and A⁻ is an anion.

10. A pharmaceutical composition comprising a compound of Formula Ia or a solvate or hydrate thereof

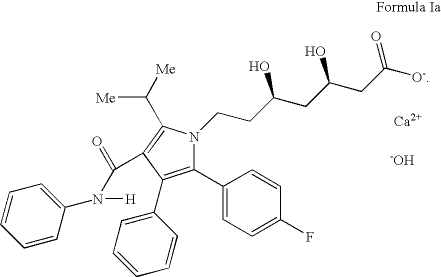

11. A method of treating hyperlipidemia, hypercholesterolemia, osteoporosis, benign prostatic hyperplasia, and Alzheimer's Disease comprising administering to a host suffering therefrom a therapeutically effective amount of a compound according to claim 1 in unit dosage form.

12. A method of treating hyperlipidemia, hypercholesterolemia, osteoporosis, benign prostatic hyperplasia, and Alzheimer's Disease comprising administering to a host suffering therefrom a therapeutically effective amount of a compound according to claim 5 in unit dosage form.

13. A method of treating hyperlipidemia, hypercholesterolemia, osteoporosis, benign prostatic hyperplasia, and Alzheimer's Disease comprising administering to a host suffering therefrom a therapeutically effective amount of a compound according to claim 6 in unit dosage form.

14. A method of treating hyperlipidemia, hypercholesterolemia, osteoporosis, benign prostatic hyperplasia, and Alzheimer's Disease comprising administering to a host suffering therefrom a therapeutically effective amount of a compound according to claim 7 in unit dosage form.

15. A process for the preparation of a mono-alkaline earth metal or zinc salt of atorvastatin or a solvate or hydrate thereof comprising:
Step (a): adding atorvastatin free acid to a solvent to form a solution;
Step (b): adding a counterion or counterions to a solvent to form a solution or suspension; and
Step (c): adding the solution from step (a) with stirring to the solution or suspension from step (b) to afford the mono-alkaline earth metal or zinc salt of atorvastatin.
